(19) United States
(12) Patent Application Publication
(10) Pub. No.: US 2021/0082556 A1
(43) Pub. Date: Mar. 18, 2021

(54) AUTOMATIC PET MEDICINE SYSTEM AND METHOD

(71) Applicant: Thomas Aaron Shaddy, Papillion, NE (US)

(72) Inventor: Thomas Aaron Shaddy, Papillion, NE (US)

(21) Appl. No.: 16/182,858

(22) Filed: Nov. 7, 2018

(51) Int. Cl. G16H 20/10; G06K 15/02; A61J 1/00; B42D 25/28; G16H 10/60; G06Q 10/08; G09F 3/02

(52) U.S. Cl. CPC G16H 20/10; G06K 15/024; A61J 1/00; B42D 25/28; G16H 10/60; G09F 2003/022; G09F 3/02; G06K 2215/0097; A61J 2205/30; G09F 2003/0202; G06Q 10/0838

(57) ABSTRACT

A system includes a memory and at least one processor to receive a photograph of a pet and store the photograph of the pet in a database, automatically determine at least one pet medicine subscription that is ready to ship based on an estimated delivery date by iterating over a list of pet medicine subscriptions, begin processing an automatic shipment associated with the at least one pet medicine subscription and retrieve shipment data associated with the at least one pet medicine subscription from the database, transmit the shipment data associated with the at least one pet medicine subscription to a server computing device, receive a shipment label from the server computing device, the shipment label associated with the shipment, and generate a personalized pet label for the at least one pet medicine subscription, the personalized pet label having the photograph of the pet.

### BACKGROUND

It can be difficult and frustrating to obtain medicine for pets. Typically, a pet owner has to schedule an appointment to visit with a veterinarian and have the veterinarian prescribe medicine for the pet. In some cases, the veterinarian may suggest that the pet owner visit a supermarket, a pet store, or a big box store and pick up an over the counter medicine. The pet owner may have to select a correct medicine for the pet. This may be difficult or confusing. In some cases, the pet owner may accidentally purchase the wrong medicine for the pet. At a minimum, this may be a waste of money. It also could be extremely dangerous for the pet if the medicine is not correct or is an incorrect dosage of the medicine.

In addition, if a pet owner owns more than one pet, it may be difficult for the owner to remember which medicine is prescribed for a particular pet. The pet owner may unintentionally give the wrong medicine to the pet. This could be problematic, dangerous, or even deadly for the pet.

It is with these issues in mind, among others, that various aspects of the disclosure were conceived.

### SUMMARY

According to one aspect, an automatic pet medicine system may include a server computing device that may execute an application such as a pet medicine application. The pet medicine application may be a web application executed by the server computing device. A client computing device may communicate with the server computing device and may transmit and request data and information from the pet medicine application.

In one example, a user may use the client computing device and create an account for use with the automatic pet medicine system. The user may register one or more pets with the automatic pet medicine system by creating a user account and providing user information such as a name, an email address, and a password. In addition, the user may provide pet information such as a type of pet (e.g., selection of a dog or a cat), a name of a pet, an age of the pet, a breed of the pet, a state of residence of the pet, and a weight of the pet. In addition, the user also may upload a photograph of the pet. The user may select a pet medicine subscription such as one of monthly or yearly. The user may setup recurring billing and be billed once per year or once a month. In another example, the user may select a one-time delivery without a subscription. The user may provide a billing address and/or a shipping address. After completing an order for the pet medicine, the server computing device may automatically generate a personalized pet label and pet medicine having the personalized pet label with the uploaded photograph of the pet may be delivered to the shipping address at a regular interval of time, e.g., once a month. The pet medicine may have an appropriate dosage for the specific pet based on the age of the pet and the weight of the pet. The pet medicine may be one of a pet medicine for a small dog (e.g., four to ten pounds), a medium dog (e.g., eleven to twenty pounds), a large dog (e.g., twenty-one to fifty-five pounds), and an extra large dog (e.g., over fifty-five pounds).

According to an aspect, a system includes a memory and at least one processor to receive a photograph of a pet and store the photograph of the pet in a database, automatically determine at least one pet medicine subscription that is ready to ship based on an estimated delivery date by iterating over a list of pet medicine subscriptions, begin processing an automatic shipment associated with the at least one pet medicine subscription and retrieve shipment data associated with the at least one pet medicine subscription from the database, transmit the shipment data associated with the at least one pet medicine subscription to a server computing device, receive a shipment label from the server computing device, the shipment label associated with the shipment, generate a personalized pet label for the at least one pet medicine subscription, the personalized pet label having the photograph of the pet, and transmit the label sheet having the personalized pet label and the shipping label.

According to another aspect, a method includes receiving, by at least one processor, a photograph of a pet and storing the photograph of the pet in a database, automatically determining, by the at least one processor, at least one pet medicine subscription that is ready to ship based on an estimated delivery date, begin processing, by the at least one processor, an automatic shipment associated with the at least one pet medicine subscription and retrieving shipment data associated with the at least one pet medicine subscription from the database, transmitting, by the at least one processor, the shipment data associated with the at least one pet medicine subscription to a server computing device, receiving, by the at least one processor, a shipment label from the server computing device, the shipment label associated with the shipment, generating, by the at least one processor, a personalized pet label for the at least one pet medicine subscription, the personalized pet label having the photograph of the pet, and transmitting, by the at least one processor, the label sheet having the personalized pet label and the shipping label.

According to an additional aspect, a non-transitory computer-readable storage medium includes instructions stored thereon that, when executed by a computing device cause the computing device to perform operations, the operations including receiving a photograph of a pet and storing the photograph of the pet in a database, automatically determining at least one pet medicine subscription that is ready to ship based on an estimated delivery date, begin processing an automatic shipment associated with the at least one pet medicine subscription and retrieving shipment data associated with the at least one pet medicine subscription from the database, transmitting the shipment data associated with the at least one pet medicine subscription to a server computing device, receiving a shipment label from the server computing device, the shipment label associated with the shipment, generating a personalized pet label for the at least one pet medicine subscription, the personalized pet label having the photograph of the pet, and transmitting the label sheet having the personalized pet label and the shipping label.

According to another aspect, a system includes a memory comprising executable instructions and at least one processor to execute the instructions to electronically receive, from an owner of a particular pet, a photograph of the particular pet and pet identifying information for the particular pet and store the photograph of the particular pet and the pet identifying information for the particular pet in a database along with information identifying the owner, automatically determine at least one pet medicine for the particular pet is to be shipped, retrieve shipment data associated with the particular pet from the database, generate a personalized pet label for the at least one pet medicine, the personalized pet label having the photograph of the particular pet and the pet identifying information for the particular pet, apply the personalized pet label to the at least one pet medicine for shipping to the owner based on the shipment data, and ship the at least one pet medicine with the personalized pet label to the owner based on the shipment data.

According to another aspect, a method includes electronically receiving, by at least one processor, from an owner of a particular pet, a photograph of the particular pet and pet identifying information for the particular pet and storing the photograph of the particular pet and the pet identifying information for the particular pet in a database along with information identifying the owner, automatically determining, by the at least one processor, at least one pet medicine for the particular pet is to be shipped, retrieving, by the at least one processor, shipment data associated with the particular pet from the database, generating, by the at least one processor, a personalized pet label for the at least one pet medicine, the personalized pet label having the photograph of the particular pet and the pet identifying information for the particular pet, applying, by the at least one processor, the personalized pet label to the at least one pet medicine for shipping to the owner based on the shipment data, and shipping, by the at least one processor, the at least one pet medicine with the personalized pet label to the owner based on the shipment data.

These and other aspects, features, and benefits of the present disclosure will become apparent from the following detailed written description of the preferred embodiments and aspects taken in conjunction with the following drawings, although variations and modifications thereto may be effected without departing from the spirit and scope of the novel concepts of the disclosure.

### BRIEF DESCRIPTION OF THE DRAWINGS

The accompanying drawings illustrate embodiments and/or aspects of the disclosure and, together with the written description, serve to explain the principles of the disclosure. Wherever possible, the same reference numbers are used throughout the drawings to refer to the same or like elements of an embodiment, and wherein:

FIG. 1 is a block diagram of a system for automatically sending pet medicine according to an example embodiment.

FIG. 2 shows a block diagram of a server computing device of the system according to an example embodiment.

FIG. 3 illustrates a flowchart for automatically sending pet medicine according to an example embodiment.

FIG. 4 illustrates another flowchart for automatically sending pet medicine according to an example embodiment.

FIG. 5 illustrates an example label sheet generated by the server computing device of the system according to an example embodiment.

FIGS. 6-18 illustrate example user interfaces of a web application displayed by a client computing device according to an example embodiment.

FIGS. 19-22 illustrate an example pet medicine box according to an example embodiment.

FIG. 23 illustrates a block diagram of a computing device according to an example embodiment.

### DETAILED DESCRIPTION

Aspects of a system and method for automatically sending pet medicine includes a user creating an account for use with the automatic pet medicine system. The user may register one or more pets with the automatic pet medicine system by providing user information such as a name, an email address, and a password. In addition, the user may provide pet information such as a type of pet (e.g., selection of a dog or a cat), a name of a pet, an age of the pet, a breed of the pet, a state of residence of the pet, and a weight of the pet. In addition, the user also may upload a photograph of the pet. The user may select a pet medicine subscription such as one of monthly or yearly. The user may be billed once per year or once a month. In another example, the user may select a one-time delivery without a subscription. The user may provide a billing address and/or a shipping address. After completing an order for the pet medicine, a server computing device may automatically generate a personalized pet label and pet medicine having the personalized pet label may be delivered to the shipping address at a regular interval of time, e.g., once a month. The pet medicine may have an appropriate dosage for the specific pet based on the age of the pet and the weight of the pet. The pet medicine may be one of a pet medicine for a small dog (e.g., four to ten pounds), a medium dog (e.g., eleven to twenty pounds), a large dog (e.g., twenty-one to fifty-five pounds), and an extra large dog (e.g., over fifty-five pounds).

In one example, the pet medicine may be Wags Advance®, which may be a medicine for repelling and killing fleas, flea eggs, ticks, and mosquitoes. In addition, the medicine may kill chewing lice and repel biting flies. Each dose of Wags Advance® may be effective for one month, but only for dogs. Wags Advance® is not suitable for use on cats. The system may automatically deliver pet medicine to the user once a month at an appropriate time, e.g., on or about every fifteenth of the month. The pet medicine may be delivered in a box having the personalized pet label including the photograph of the pet and optionally pet identifying information such as one or more of the name of the pet, the weight of the pet, the breed of the pet, and/or the age of the pet.

Conventionally, flea and tick topical treatments are dosed by weight of a pet. Sometimes products have a generic picture of a dog on the packaging. If a customer has multiple pets in the same home, they could accidentally mistake the appropriate treatment for a particular dog. This can cause an adverse event (e.g., if an extra large dog treatment is applied to a small dog) or an under-dosing event (if a small dog treatment is applied to an extra large dog). This problem is amplified because traditional selling units are multiple doses per carton.

As an example, a pet owner may own two dogs including Cujo (an extra large dog) and Fluffy (a small dog). The pet owner may visit a retail store or order from an online retailer and purchase a three month supply for both dogs. The pet owner may apply the correct dose to each dog for the first month and throw away the packaging. The next month, the pet owner may apply the incorrect dose to each dog. This could harm the pets. Cujo may receive an inappropriate underdose and suffer from fleas. Fluffy may receive an inappropriate overdose and may become ill.

In another example, a pet owner may own a dog and a cat. If the pet owner were to accidentally apply the medicine designed for the dog to the cat, this could severely harm the cat.

The system discussed herein solves these problems by automatically delivering a single month's dose in a box having a personalized pet label on the box including a photograph of the pet and optionally pet identifying information, such as one or more of a name of the pet, a weight of the pet, a breed of the pet, and/or an age of the pet. The pet owner has a visual reference that allows the pet owner to apply the correct dose to the correct pet. The next month, the system automatically sends the next dose so that it is scheduled to arrive on or before the day the pet owner should apply the dose. Again, the box includes the personalized pet label on the box including the photograph of the pet and optionally pet identifying information, such as the name of the pet.

The system may include a memory and at least one processor to receive an uploaded photograph of a pet and store the uploaded photograph of the pet in a database, automatically determine at least one pet medicine subscription that is ready to ship based on an estimated delivery date by iterating over a list of pet medicine subscriptions, begin processing an automatic shipment associated with the at least one pet medicine subscription and retrieve shipment data associated with the at least one pet medicine subscription from the database, transmit the shipment data associated with the at least one pet medicine subscription to a server computing device, receive a shipment label from the server computing device, the shipment label associated with the shipment, generate a personalized pet label for the at least one pet medicine subscription, the personalized pet label having the uploaded photograph of the pet and optionally pet identifying information, such as one or more of a name of the pet, a weight of the pet, a breed of the pet, and/or an age of the pet, and transmit the label sheet having the personalized pet label and the shipping label to a recipient. In another example, the system transmits a Portable Document Format (PDF) file of the personalized pet label, an image of the personalized pet label, or another file with the personalized pet label to a computing device instead of or in addition to transmitting the personalized pet label to the recipient

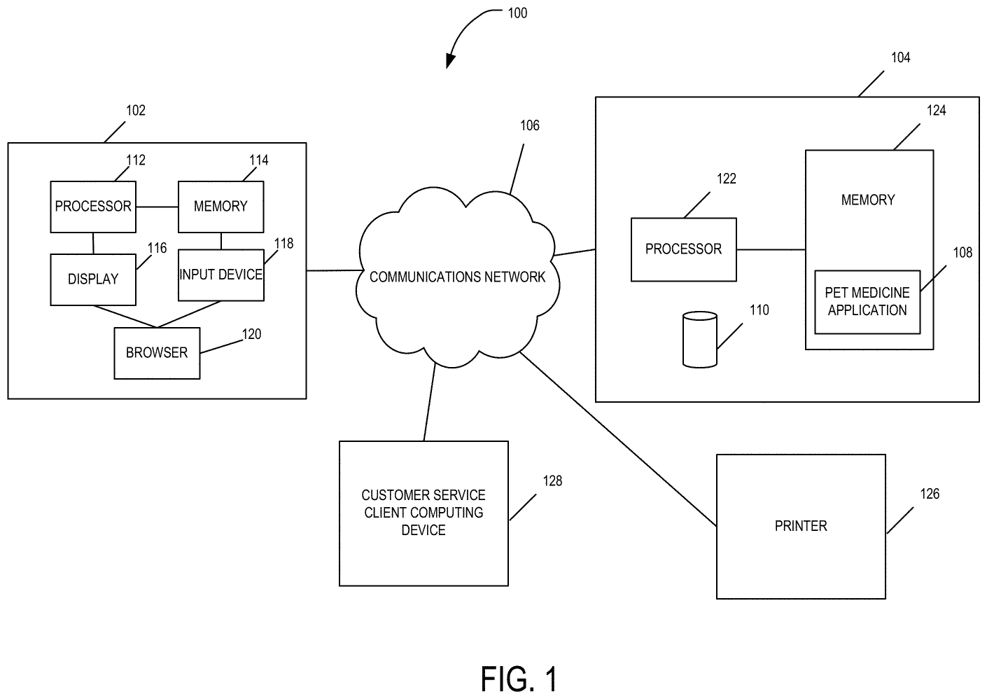

FIG. 1 shows a block diagram of a computing system comprising an automatic pet medicine system 100 according to an example embodiment. The automatic pet medicine system 100 includes at least one client computing device 102 that may be in communication with at least one server computing device 104 via a communication network 106. The automatic pet medicine system 100 may also include a relational database management system (RDBMS) or another type of database management system that stores and communicates data from at least one database 110. The data stored in the at least one database 110 may be user information associated with each user and each user account such as username information, password information, shipping information, billing information, subscription information (e.g., yearly subscription or monthly subscription) and pet information (optionally including pet identifying information) associated with each pet associated with each user, such as a name of a pet, a breed of the pet, a weight of the pet, a photograph of the pet, and an age of the pet, among other information. The subscription is not limited to a yearly subscription or a monthly subscription. As an example, the subscription may be a multi-year subscription or a twice-a-month subscription if the pet medicine is to be applied more than once a month unlike Wags Advance. In another example, the user may select a one-time delivery without a subscription. In addition, the database 110 also may store other information such as order history information among other information.

The at least one client computing device 102 is configured to receive data from and/or transmit data to the at least one server computing device 104 through the communication network 106. Although the at least one server computing device 104 is shown as a single server it is contemplated that the at least one server computing device 104 may include multiple servers such as in a cloud computing configuration. The client computing device 102 may be used, for example, by an owner of one or more pets to transmit photographs of the one or more pets, pet identifying data of the one or more pets, subscription data, shipment data for one or more medicines for the one or more pets, and/or other data of the owner to the server computing device 104.

The at least client computing device 102 includes at least one processor 112 to process data and memory 114 to store data. The processor 112 processes communications, builds communications, retrieves data from memory 114, and stores data to memory 114. The processor 112 and the memory 114 are hardware. The memory 114 may include volatile and/or non-volatile memory, e.g., a computer-readable storage medium such as a cache, random access memory (RAM), read only memory (ROM), flash memory, or other memory to store data and/or computer-readable executable instructions such as browser application that may display a user interface provided by a pet medicine application 108 executed by the server computing device 104. In addition, the at least one client computing device 102 further includes at least one communications interface to transmit and receive communications, messages, and/or signals.

The at least one client computing device 102 can be a laptop computer, a smartphone, a personal digital assistant, a tablet computer, a standard personal computer, or another processing device. The at least one client computing device 102 may include a display 116, such as a computer monitor, for displaying data and/or graphical user interfaces. The at least one client computing device 102 may also include an input device 118, such as a camera for capturing a photograph of a pet, a keyboard or a pointing device (e.g., a mouse, trackball, pen, or touch screen) to enter data into or interact with graphical and/or other types of user interfaces. In an exemplary embodiment, the display 116 and the input device 118 may be incorporated together as a touch screen of the smartphone or tablet computer.

The at least one client computing device 102 may display on the display 116 a graphical user interface (or GUI) to generate a graphical user interface on the display 116. The graphical user interface may be provided by the pet medicine application 108 and displayed by a browser on the display 116. The graphical user interface enables a user of the at least one client computing device 102 to interact with the pet medicine application 108.

The at least one server computing device 104 includes at least one processor 122 to process data and memory 124 to store data. The processor 122 processes communications, builds communications, retrieves data from memory 124, and stores data to memory 124. The processor 122 and the memory 124 are hardware. The memory 124 may include volatile and/or non-volatile memory, e.g., a computer-readable storage medium such as a cache, random access memory (RAM), read only memory (ROM), flash memory, or other memory to store data and/or computer-readable executable instructions such as a portion or a component of the pet medicine application 108. In addition, the at least one client computing device 104 further includes at least one communications interface to transmit and receive communications, messages, and/or signals.

The communication network 106 can be the Internet, an intranet, or another wired or wireless communication network. For example, the communication network 106 may include a Mobile Communications (GSM) network, a code division multiple access (CDMA) network, 3^{rd} Generation Partnership Project (GPP) network, an Internet Protocol (IP) network, a wireless application protocol (WAP) network, a WiFi network, a Bluetooth network, a satellite communications network, or an IEEE 802.11 standards network, as well as various communications thereof. Other conventional and/or later developed wired and wireless networks may also be used.

The pet medicine application 108 may be a component of an application and/or service executable by the at least one server computing device 104 and/or the at least one client computing device 102. For example, the pet medicine application 108 may be a single unit of deployable executable code or a plurality of units of deployable executable code. According to one aspect, the pet medicine application 108 may include one component that may be a web application, a native application, and/or a mobile application (e.g., an app) downloaded from a digital distribution application platform that allows users to browse and download applications developed with mobile software development kits (SDKs) including the App Store and GOOGLE PLAY®, among others.

The automatic pet medicine system 100 may include one or more customer service client computing devices 128 that are connected to the communications network 106. In addition, the automatic pet medicine system 100 may include one or more printers 126 that are connected to the communications network 106. The personalized pet label and the shipping label may be transmitted to the one or more customer service client computing devices 128 and/or the printer 126 and printed. The personalized pet label and the shipping label may be applied to a pet medicine box and a shipping box and the shipping box may be shipped to a recipient.

The printer 126 includes at least one processor to process data and memory to store data. The processor processes communications, builds communications, retrieves data from memory, and stores data to memory. The processor and the memory are hardware. The memory may include volatile and/or non-volatile memory, e.g., a computer-readable storage medium such as a cache, random access memory (RAM), read only memory (ROM), flash memory, or other memory to store data and/or computer-readable executable instructions such as the pet medicine application 108 executed by the server computing device 104. In addition, the printer 126 further includes at least one communications interface to transmit and receive communications, messages, and/or signals.

The customer service client computing device 128 includes at least one processor to process data and memory to store data. The processor processes communications, builds communications, retrieves data from memory, and stores data to memory. The processor and the memory are hardware. The memory may include volatile and/or non-volatile memory, e.g., a computer-readable storage medium such as a cache, random access memory (RAM), read only memory (ROM), flash memory, or other memory to store data and/or computer-readable executable instructions such as a browser application that may display a user interface provided by the pet medicine application 108 executed by the server computing device 104. In addition, the customer service client computing device 128 further includes at least one communications interface to transmit and receive communications, messages, and/or signals.

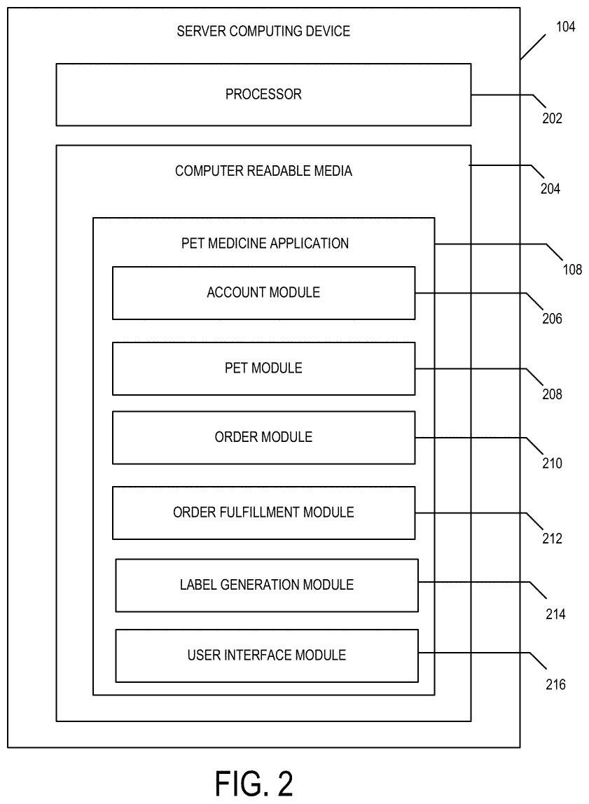

FIG. 2 illustrates a block diagram of the server computing device 104 according to an example embodiment. The server computing device 104 includes at least one processor 202 and computer readable media (CRM) 204 in memory on which the pet medicine application 108 or other user interface or application is stored. The computer readable media 204 may include volatile media, nonvolatile media, removable media, non-removable media, and/or another available medium that can be accessed by the processor. By way of example and not limitation, the computer readable media 204 comprises computer storage media and communication media. Computer storage media includes non-transitory storage memory, volatile media, nonvolatile media, removable media, and/or non-removable media implemented in a method or technology for storage of information, such as computer/machine-readable/executable instructions, data structures, program modules, or other data. Communication media may embody computer/machine-readable/executable instructions, data structures, program modules, or other data and include an information delivery media or system, both of which are hardware.

The pet medicine application 108 includes an account module 206 for creating a user account associated with the system 100. As an example, a user may create an account for use with the system by providing name information (e.g., a first name and a last name), email address information, and password information. In one example, the user may enter the name information, email address information, and password information into a user interface displayed by the client computing device 102. The user of the client computing device 102 may select a confirm user interface element and the client computing device 102 may transmit the name information, the email address information, and the password information to the server computing device 104. The server computing device 104 may store a representation of the name information, the email address information, and password information in the database 110. In one aspect, the user is an owner of one or more pets.

In addition, the account module 206 may allow the user to edit and/or modify the account information. The user of the client computing device 102 may edit and/or modify account information such as the name information, email address information, and password information.

The pet medicine application 108 may include a pet module 208 for associating information for one or more pets with a user account associated with the system 100 or for otherwise associating information for one or more pets to an owner of the one or more pets and the owner's information. As an example, after creating the user account, the user may be asked to provide information associated with one or more pets. In this example, the user is the owner of the one or more pets. The user of the client computing device may select a type of a pet (e.g., a dog or a cat) and may transmit the selection of the type of the pet to the pet module 208 of the pet medicine application 108. Next, the user may be asked to provide pet information that allows the pet medicine application 108 to select the appropriate pet medicine for the pet. The user may provide pet information, including pet identifying information, such as a name of the pet (e.g., Old Yeller), an age of pet (e.g., twenty years old), a breed of the pet (e.g., Labrador Retriever), a state of residence of the pet (e.g., Texas), and/or a weight of the pet (e.g., fifty-five pounds or greater). In addition, the user may optionally upload a photograph of the pet to the server computing device 104. The client computing device 102 may transmit the photograph of the pet to the server computing device 104 along with the pet information and the server computing device 104 may store a representation of the pet information, including the photograph and the pet identifying information, in the database 110. For example, the server computing device 104 may store the pet information in the database 110 in a way that it is associated with the user/owner of the pet, such as by storing it with or indexed by name, address, email address, username, account name, account number, or other user/owner identifying information. If the user does not have a photograph of the pet available, the user could choose to upload or transmit the photograph of the pet to the server computing device 104 at a later time. The user of the client computing device 102 may then repeat these steps for one or more other pets of the user. In another example, the one or more pets are owned by someone other than the user.

If the user does not upload a photograph of the pet, then the pet module 208 may select a photograph of a breed of the pet from a database of example photographs that may be stored in the database 110. Each breed in the list of breeds may have an example photograph stored in the database 110. This photograph of the breed of the pet may be used as a placeholder for the pet.

The pet medicine application 108 may include an order module 210 that allows a user to place an order for pet medicine for a particular pet associated with the user account. As an example, based on the pet information, the order module 210 may select an appropriate medicine for the particular pet. The appropriate medicine may be based on the age of the pet and the weight of the pet. As an example, the medicine may not be suitable for pets under a certain age and/or over a certain age. In addition, the pet medicine dosage may be determined based on the weight of the pet.

The pet medicine may be, for example, Wags Advance for a small dog (e.g., four to ten pounds), a medium dog (e.g., eleven to twenty pounds), a large dog (e.g., twenty-one to fifty-five pounds), or an extra large dog (e.g., over fifty-five pounds). Wags Advance may include Imidacloprid, Permethrin, and Pyriproxyfen and may be included in a tube device in a pet medicine box. These are the same active ingredients found in K9 ADVANTIX® II for dogs. Wags Advance may be a pet medicine that may be applied to the pet. The application of the pet medicine may be effective for one month or a different period of time. However, in another embodiment, the pet medicine may be different from Wags Advance and/or for another duration or period of time, such as a number of days. The user also may place an order for a product in addition to the pet medicine and/or other than pet medicine that may be delivered to a customer at a regular interval of time, such as monthly or bi-monthly. The product other than the pet medicine may be a pet-related product such as pet food or pet treats. However, the product may not be a pet-related product.

Wags Advance may be a medicine for repelling and killing fleas, flea eggs, ticks, and mosquitoes. In addition, the medicine may kill chewing lice and repel biting flies. As a result, the system may automatically deliver pet medicine to the user once a month at an appropriate time based on a previous delivery date, if applicable. The pet medicine may be delivered in a box having the personalized pet label including the photograph of the pet and the pet identifying information, including the name of the pet, the weight of the pet, the breed of the pet, and/or the age of the pet. In addition, the box may have a particular label associated with the size of the dog and may have a color associated with the size of the dog, among other features.

The user may order the pet medicine by providing credit card information (e.g., a credit card number, an expiration date, a card verification value (CVV), and a name on the card), a billing address, and a shipping address if the shipping address is different than the billing address. The user may select a complete order user interface element and the order module 210 may begin processing the order. In one example, the order may be placed into a queue and scheduled to be fulfilled once a month on a particular day. In another embodiment, the order may be scheduled to be fulfilled at a different regular interval of time such as twice a month or once every two months.

If the user moves to a new address or would like the order to be delivered to a different location, the user can modify the shipping address and/or the billing address. The user may submit their username and password to authenticate with the pet medicine application 108 and may use a user interface provided by the pet medicine application to edit the shipping address and/or the billing address. The user may edit user interface elements that display a current shipping address and/or a current billing address and may modify the shipping address and/or the current billing address and select a user interface element that when selected, transmits the updated shipping address and/or the updated billing address to the server computing device 104. The server computing device 104 receives the updated shipping address and/or the updated billing address and stores the data in the database 110 associated with the other user information. In addition, the user also may modify the credit card information in a similar manner to use a different credit card and/or if the credit card expires.

In another example, the user could update the weight of the pet if the weight were to change, e.g., as the pet matures from a puppy to an adult dog. The server computing device 104 also may send automatic notifications to the owner of the pet in certain instances. For example, if the pet was a puppy when the owner created the account associated with the pet, the pet medicine application 108 can automatically send the owner a notification requesting that the owner check the weight of the pet at a later date. If the weight is incorrect or outdated, the owner or user can modify the weight of the pet.

The pet medicine application 108 may include an order fulfillment module 212 that executes a particular job at a particular interval of time, e.g., once a day on business days at 7:00 AM in a particular time zone (e.g., Central Time Zone). In one example, the order fulfillment module 212 may evaluate a plurality of pending and scheduled orders and determine at least one order that is scheduled to be fulfilled on that day. This may include at least one order for pet medicine that is to be delivered to a particular shipping address. In one example, the order fulfillment module 212 may determine that the at least one order is scheduled to be fulfilled based on a delivery date for the order. As an example, the user may have ordered the medicine on October 1. The order fulfillment module 212 may schedule the delivery of the medicine for three days after the initial order, e.g., October 4. As long as the user has an active subscription, the order fulfillment module 212 may schedule the delivery of the medicine on the first of each month to be delivered by the fourth of the month. In another example, the order fulfillment module 212 may schedule the delivery of the medicine each month on the anniversary of the purchase of the subscription, e.g., the fifteen of the month. “At least one” as used herein means one or more.

The at least one order that is scheduled to be fulfilled on that day may be assigned a same manifest identifier. This manifest identifier may make it easier for a package delivery service (e.g., the United States Postal Service) to pick up all of the shipments in a single scan.

As an example, a user (e.g., a customer or pet owner) may have one designated ship day cycle per month. The order fulfillment module 212 may consolidate all individual subscriptions (e.g., products per pet) into a single shipment each month. As discussed herein, when a new user creates an account, the user may add any number of pets to the account and specify a name of each pet, an age of each pet, a breed of each pet, a weight of each pet, and may optionally provide a photograph of each pet. After submitting payment for the subscriptions, the subscription ship day may be selected based on a billing date.

If a user adds additional pets before a first order delivery is processed, then the user may be charged immediately for any new subscriptions and the subscriptions may be available for shipment with the first shipment. However, if the user adds additional pets after the first order delivery is processed, then the user may not be charged immediately. The subscription payment may be queued until a next ship day cycle. On that date, new subscriptions may be paid for and made deliverable along with existing subscriptions. Any subscriptions with a monthly billing interval may be added to the existing monthly subscriptions. In addition, any subscriptions with a yearly billing interval may begin with a new yearly subscription that may renew on the date in a following year.

According to an example embodiment, a user may receive a payment notification from the order fulfillment module 212 when a payment method charged (e.g., a credit card). This may occur when the user first places an order, if additional subscriptions are added before a first shipment, and when payment is processed based on the billing interval (e.g., monthly or yearly). However, if a user's payment method fails for a first order associated with a new subscription, the user may be immediately alerted and given the opportunity to provide a different payment method. If a user's payment method fails on a subscription renewal, the user may be sent a message (e.g., an email or a push notification) that explains issues and/or problems processing payment along with information associated with the payment method. Payment may be attempted again three days later. If the payment fails again, another message may be sent to the user. Payment may be attempted again three days after the second failed payment. If that payment fails, the subscription may be canceled.

In other words, a first user may register with the pet medicine application 108 and create an account on January 1. The first user may have one pet dog named Alpo, a small dog. On January 2 at 5:00 PM, the user may order pet medicine for Alpo and may have signed up for a yearly subscription. This order may be placed into a queue of orders to be processed by the order fulfillment module 212. On January 3 at 7:00 AM, this order may be processed by the order fulfillment module 212 for delivery to the first user by January 6. After processing this order, the order fulfillment module 212 may automatically schedule the next order for February 3 and place the next order into the queue of orders. On February 3 at 7:00 AM, the order may be processed by the order fulfilment module 212 for delivery to the first user by February 6. This process may continue every month for up to one year from when the yearly order was placed.

A second user may register with the pet medicine application 108 and create an account on December 31. The second user may have two pet dogs. The first dog associated with the second user may be named Biscuit, a medium dog. The second dog associated with the second user may be named Cooper, a large dog. On January 1 at 6:00 AM, the user may order pet medicine for Biscuit and Cooper. This order may be placed into the queue of orders to be processed by the order fulfillment module 212. The job to process the plurality of pending and scheduled orders may not be executed on January 1 at 7:00 AM because it is a federal holiday. On January 2 at 7:00 AM, this order may be processed by the order fulfillment module 212 for delivery to the second user by January 5. After processing this order, the order fulfillment module 212 may schedule the next order for February 2 and place the order into the queue of orders. On February 2 at 7:00 AM, the order may be processed by the order fulfillment module 212 for delivery to the second user by February 5.

The pet medicine application 108 may include a label generation module 214 that generates labels for the at least one order that is scheduled to be fulfilled. As an example, for the at least one order, the label generation module 214 may retrieve shipment data associated with each order scheduled to be fulfilled and transmit the shipment data to another server computing device. In one example, the shipment data may include a weight of the shipment based on a number of pet medicines to be sent in the shipment, a billing address for the shipment, a shipping address for the shipment, and other information. In one example, a billing address for a shipment, a shipping address for a shipment, and other shipment data and information is for an owner of one or more pets for which a medicine is shipped. In one example, a billing address for a shipment, a shipping address for a shipment, and other shipment data and information is associated with each particular pet for which a particular medicine is shipped.

In one example, the shipment data may be sent using a web application programming interface (API) and may be sent to a server associated with SHIPPO™ or another shipping provider. The server computing device may generate a shipping label and transmit the shipping label to the label generation module 214. In addition, the label generation module 214 may retrieve the pet information associated with the order including a photograph of the pet and the pet identifying information, such as the pet name, the pet age, the breed of the pet, and/or the pet weight. The label generation module 214 may generate a personalized pet label having the photograph of the pet and the pet identifying information, including the pet name, the pet age, the breed of the pet, and/or the pet weight, among other information.

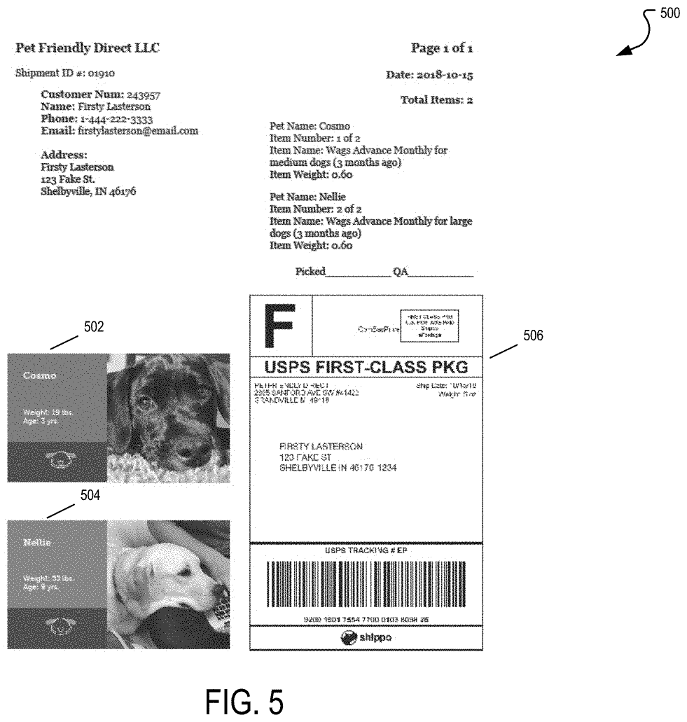

In some cases, if the user has more than one pet and has ordered medicine for more than one pet, there may be more than one personalized pet label for the order, e.g., one medicine for each pet. The label generation module 214 may then generate a label sheet having the shipping label for the order and the at least one personalized pet label. In one example, the label sheet may be an image file, a portable document format (PDF) file, or another type of file. The label sheet may be transmitted to a recipient for processing. As an example, the label sheet may be sent via email to the recipient, such as the customer service client computing device 128. An example of a label sheet is shown in FIG. 5.

The label sheet may include other additional information such as a shipment identifier associated with the order, a customer number, a customer name, a customer phone number, a customer email address, and a customer address, and item information for the order, among other information. In one example, the recipient may be a member of a customer service team. The customer service team may have the customer service client computing device 128 and receive the label sheet using the customer service client computing device 128.

The customer service team may print a copy of the label sheet using the printer 126 and may attach the shipping label and the personalized pet label to a medicine box. In another example, the label generation module 214 may automatically transmit the label sheet to the printer 126. The printer 126 may print the label sheet for use.

The shipping label may be a first label that may be affixed to a shipping box and the personalized pet label may be a second label that may be affixed to the medicine box that is placed inside of the shipping box and shipped using a courier. The medicine box and the shipping box may be associated with the manifest identifier. In addition, the medicine box and the shipping box may be associated with a tracking identifier that is specific to the medicine box and the shipping box.

In one example, a daily job may include two shipments. In another example, the daily job may include one thousand shipments. A first shipment may include a first medicine box for a first pet and a second medicine box for a second pet. Both the first medicine box and the second medicine box are being shipped to a first address. A second shipment may include a third medicine box for a third pet and a fourth medicine box for a fourth pet. Both the third medicine box and the fourth medicine box are being shipped to a second address. Additional shipments may include similar components: one or more medicine boxes included in a shipping box.

In addition, the pet medicine application 108 includes a user interface module 216 for transmitting a user interface to the client computing device 102 to be displayed on the display 116. As an example, the user interface module 216 generates a native and/or web-based graphical user interface (GUI) that accepts input and provides output viewed by users of the client computing device 102. The client computing device 102 may provide realtime automatically and dynamically refreshed pet medicine information. The user interface module 216 may send data to other modules of the pet medicine application 108 of the server computing device 104, and retrieve data from other modules of the pet medicine application 108 of the server computing device 104 asynchronously without interfering with the display and behavior of the user interface displayed by the client computing device 102.

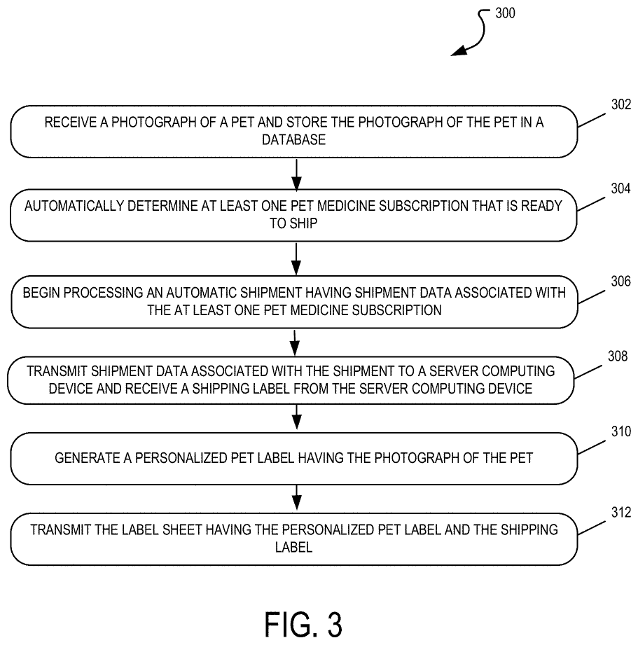

FIG. 3 illustrates a flowchart of a process 300 for automatically sending pet medicine according to an example embodiment. In step 302, as noted above, when a user creates a user account, the user is asked to associate at least one pet with the user account. The client computing device 102 may transmit the photograph of a pet to the server computing device 104 along with the pet information and the server computing device 104 may store a representation of the pet information including the photograph in the database 110. If the user does not have a photograph of the pet available, the user could choose to upload the photograph of the pet at a later time.

If the user does not upload a photograph of the pet, then the pet module 208 may select a photograph of a breed of the pet from a database of example photographs that may be stored in the database 110. Each breed in the list of breeds may have an example photograph stored in the database 110. This photograph of the breed of the pet may be used as a placeholder for the pet.

In step 304, the order fulfillment module 212 of the pet medicine application 108 may execute the particular job at the particular time, e.g., at 7:00 AM on each business day. When executing the job, the pet medicine application 108 may iterate over each user and each subscription associated with each user of the system 100 and determine whether medicine associated with each subscription is to be shipped based on an estimated delivery date. At this time, the order fulfillment module 212 may determine at least one pet medicine subscription that is ready to ship. The at least one pet medicine subscription may be determined to be ready to ship based on the delivery date for the order. As an example, the at least one pet medicine subscription may be scheduled to be delivered in three days.

Next, in step 306, the order fulfillment module 212 may determine a shipment having shipment data associated with the at least one pet medicine subscription. The order fulfillment module 212 may retrieve shipment data associated with the at least one pet medicine subscription. More than one pet medicine subscription may be associated with a shipment, e.g., two dogs may reside at a particular shipping address.

In step 308, the label generation module 214 may transmit the shipment data associated with the shipment to a server computing device and may receive a shipping label from the server computing device. The shipping label may be prepaid and have a shipping address for the shipment and be for the United States Postal Service (USPS) or another shipping courier. In another embodiment, the label generation module 214 may generate the shipping label.

In step 310, the label generation module 214 may generate at least one personalized pet label and arrange the at least one personalized pet label and the shipping label on a label sheet. Again, if the user has two pets and each have a subscription for pet medicine, the label sheet may include a first personalized pet label for a first pet, a second personalized pet label for a second pet, and a shipping label. Each personalized pet label may include a photograph of the pet and pet identifying information, such as a name of the pet, a weight of the pet, the breed of the pet, and/or an age of the pet, among other information.

In step 312, the label generation module 214 may transmit the label sheet to a recipient computing device such as the customer service client computing device 128. In one example, the label generation module 214 may automatically email the label sheet to a particular email address, such as a customer service email address associated with the pet medicine application 108. The label sheet may be a PDF or another type of file or document. The label sheet also may be part of a pick sheet that may be used for order picking or order preparation of the shipment.

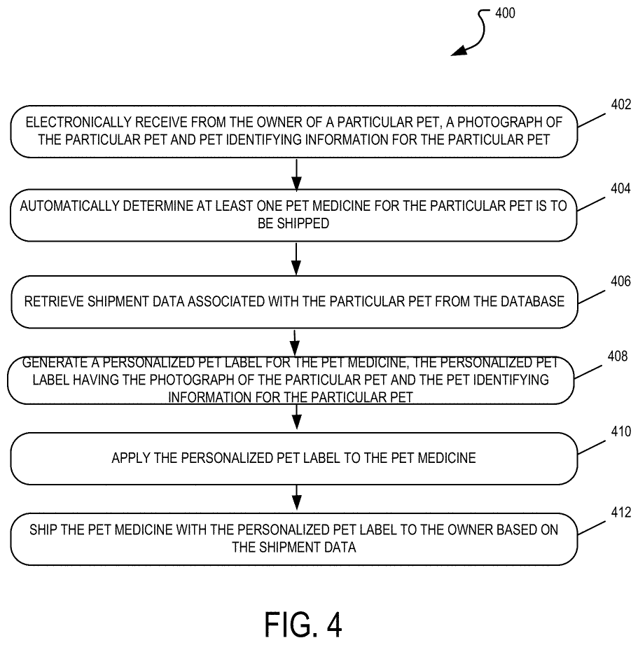

FIG. 4 illustrates another flowchart of a process 400 for automatically sending pet medicine to an owner of a pet according to an example embodiment. In step 402, as noted above, when a user, such as an owner of a pet, creates a user account, the user is asked to associate at least one pet with the user account. The owner of the pet may use the client computing device 102 to provide a photograph of a particular pet and pet identifying information for the particular pet. The pet identifying information for the particular pet may include one or more of a pet name, a pet breed, a pet weight, a pet age, and an address of the pet. The client computing device 102 may transmit the photograph of the particular pet and the pet identifying information to the server computing device 104. The server computing device 104 may receive the photograph of the particular pet and the pet identifying information and store the photograph of the particular pet and the pet identifying information in the database 110 along with information that identifies the owner.

In step 404, order fulfillment module 212 of the pet medicine application 108 executed by the server computing device 104 may automatically determine that at least one pet medicine for the particular pet is to be shipped. Next, in step 406, the order fulfillment module 212 of the pet medicine application 108 may retrieve shipment data associated with the particular pet from the database 110. In step 408, the label generation module 214 of the pet medicine application 108 may generate a personalized pet label for the at least one medicine. The personalized pet label may have the photograph of the particular pet and the pet identifying information for the particular pet.

In step 410, the personalized pet label may be applied to the at least one pet medicine. In one example, the personalized pet label may be applied to the medicine box. In one example, the personalized pet label may be applied to the medicine box by a member of the customer service team. In another example, the personalized pet label may be applied to the medicine box by a computing device such as the server computing device 104, the printer 126, or the customer service client computing device 128. In step 412, the at least one pet medicine with the personalized pet label may be shipped to the owner based on the shipment data. In one example, a member of the customer service team may ship the at least one pet medicine with the personalized pet label. In another example, the server computing device 104, the printer 126, and/or the customer service client computing device 128 may initiate the shipment by sending a notification to a courier.

In a further example, the owner may have a plurality of pets. The owner may use the client computing device 102 to provide a photograph corresponding to each pet of the plurality of pets and pet identifying information for each pet of the plurality of pets. The server computing device 104 may receive the photograph of each pet of the plurality of pets and the pet identifying information for each pet of the plurality of pets and store the photograph of each and pet and the pet identifying information for each pet in the database 110 along with information that identifies the owner. The server computing device 104 may automatically determine at least one pet medicine for each pet that is to be shipped and retrieve shipment data associated with each pet from the database 110. The server computing device 104 may generate a personalized pet label for each pet medicine. Each personalized pet label may have the photograph and the pet identifying information for a corresponding pet. In addition, the personalized pet label may be applied to the pet medicine for the corresponding pet and each pet medicine may be shipped with the corresponding pet label to the owner based on the shipment data.

FIG. 5 shows an example label sheet 500 or pick sheet according to an example embodiment. As shown in FIG. 5, the label sheet 500 includes a first personalized pet label 502 for Cosmo and a second personalized pet label 504 for Nellie. In addition, the label sheet also includes a shipping label 506. As shown in FIG. 5, the shipping label is a prepaid USPS shipping label with a shipping address: Firsty Lasterson 123 Fake St. Shelbyville, Ind. 46176. The label sheet 500 also includes other information such as a customer number of 243957 and a shipment identifier of 01910. The label sheet also includes information associated with each pet medicine in the shipping box including a pet name for the first personalized pet label, an item number, an item name, and an item weight. The label sheet 500 may also include areas that may allow the customer service team to confirm that the shipment has been picked and been reviewed by quality assurance.

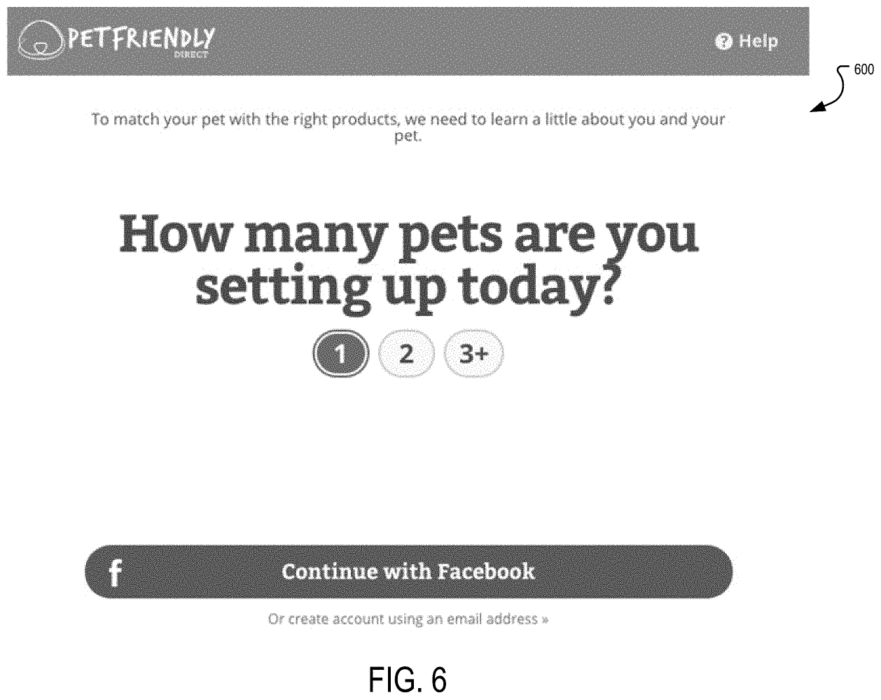

FIG. 6 shows an example user interface 600 generated by the pet medicine application 108 and transmitted from the server computing device 104 to the client computing device 102 and displayed by the client computing device 102. In one example, the pet medicine application 108 may be a web application and displayed by the browser of the client computing device 102. As shown in FIG. 6, the user is asked to indicate how many pets are being associated with the user account. The user may select one pet, two pets, or three or more pets. Other options are possible.

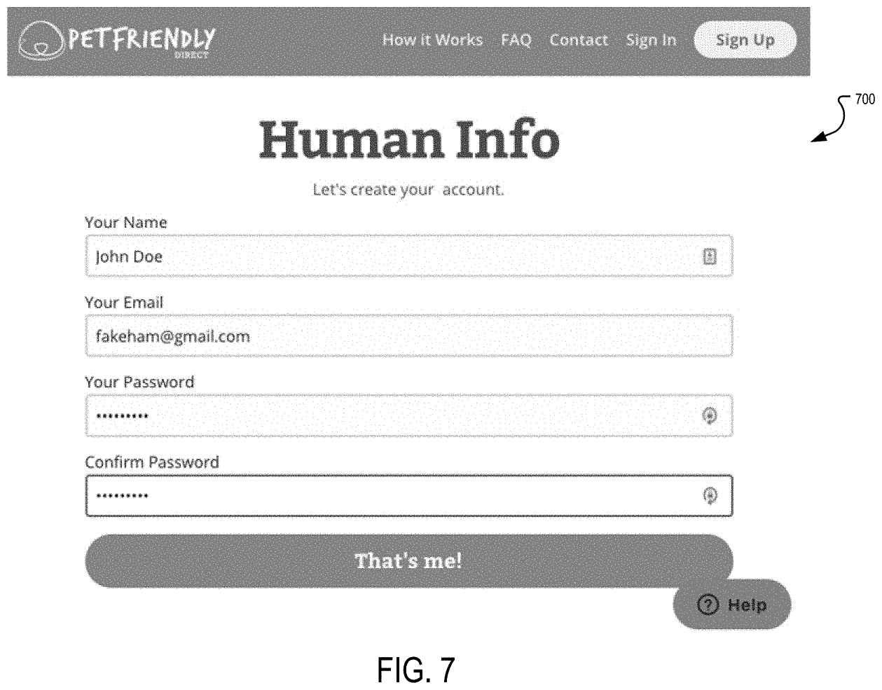

FIG. 7 shows an example user interface 700 generated by the pet medicine application 108 and transmitted from the server computing device 104 to the client computing device 102 and displayed by the client computing device 102. As shown in FIG. 7, the user provides user account information including a name, an email address, and a password. The user is requested to provide the password twice to confirm that the password is correct. This email address and password may be used to authenticate the user and log in to the pet medicine application 108. The client computing device may transmit a representation of the name, the email address, and the password to the server computing device 104. The server computing device 104 may receive the representation and store the representation in the database 110 as a new user account.

As another example, the user may register with the system 100 using FACEBOOK™ Login. This may allow the user to use their previously existing FACEBOOK™ account including their username and password that they may use to login into FACEBOOK™ to login to the system 100. FACEBOOK™ Login provides a fast and convenient way for users to create an account for use with the system 100 and log in to the pet medicine application 108. FACEBOOK™ Login may be used to authenticate the user and may allow the user to use their FACEBOOK™ password to log in to the pet medicine application 108.

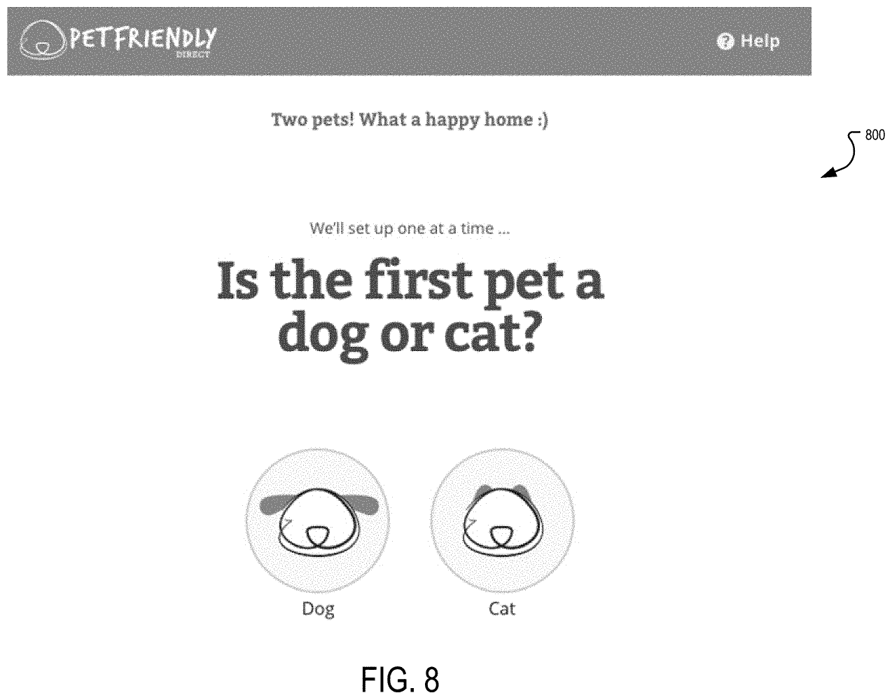

FIG. 8 shows an example user interface 800 generated by the pet medicine application 108 and transmitted from the server computing device 104 to the client computing device 102 and displayed by the client computing device 102. As shown in FIG. 8, the user selects a pet type for a pet associated with the user account, e.g., a dog or a cat. Other options are possible.

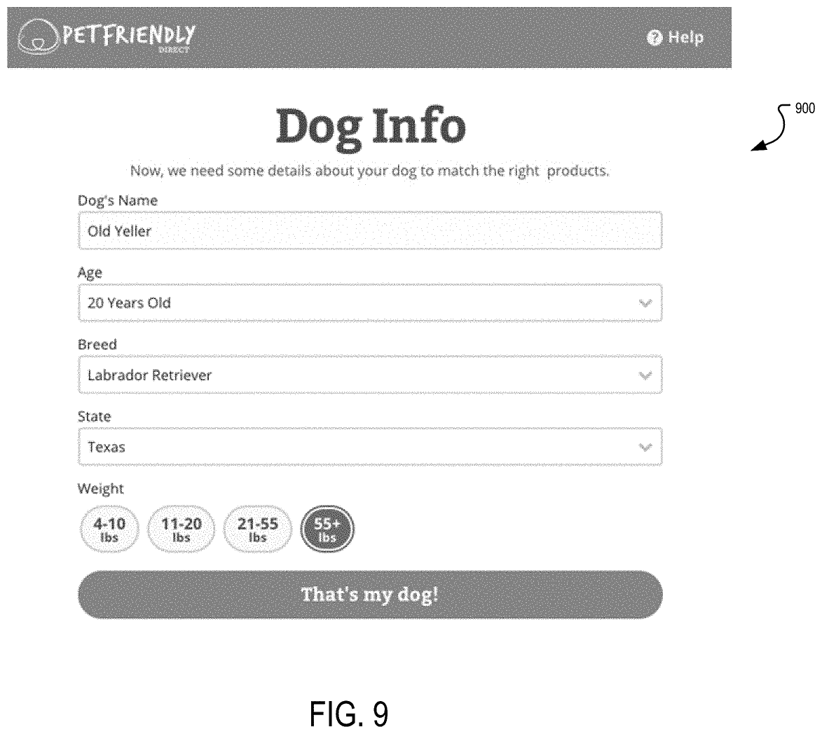

FIG. 9 shows an example user interface 900 generated by the pet medicine application 108 and transmitted from the server computing device 104 to the client computing device 102 and displayed by the client computing device 102. As shown in FIG. 9, the user provides pet information associated with the pet. In this case, the pet is a dog.

The dog information may include a dog's name, an age of the dog, a breed of the dog selected from a list of breeds, a state of residence of the dog, and/or a weight of the dog (e.g., four to ten pounds, eleven to twenty pounds, twenty-one pounds to fifty-five pounds, and greater than fifty-five pounds). After the user provides the dog information, the client computing device 102 may transmit the dog information to the server computing device 104 and the server computing device may store the dog information in the database 110 associated with the user account.

In another example, the pet may be a cat. The user may provide cat information including a cat's name, an age of the cat, a breed of the cat selected from a list of breeds, a state of residence for the cat, and/or a weight of the cat.

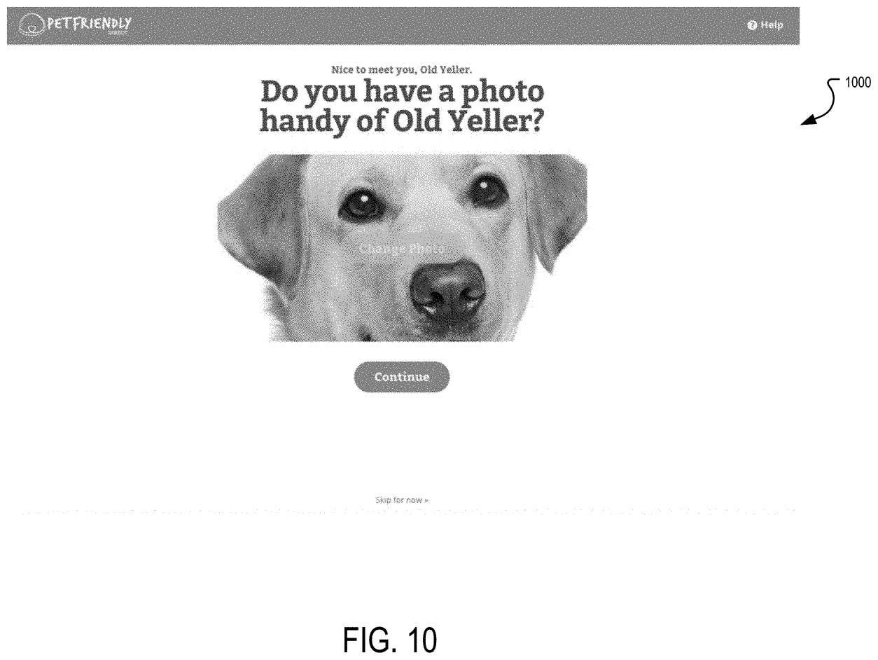

FIG. 10 shows an example user interface 1000 generated by the pet medicine application 108 and transmitted from the server computing device 104 to the client computing device 102 and displayed by the client computing device 102. As shown in FIG. 10, the user may select and upload a photograph of the pet. In this case, the user selects and uploads a photograph of Old Yeller, a Labrador Retriever. The user may select a portion or a subset of the photograph as uploaded to represent the pet. The client computing device 102 may transmit the photograph to the server computing device 104 and the server computing device 104 may store the photograph along with the dog information in the database 110.

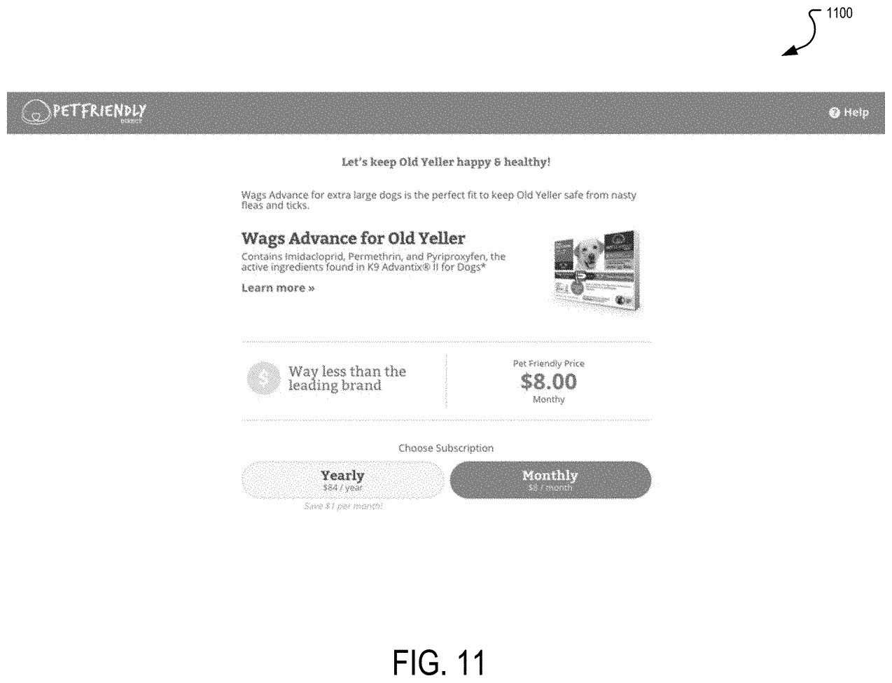

FIG. 11 shows an example user interface 1100 generated by the pet medicine application 108 and transmitted from the server computing device 104 to the client computing device 102 and displayed by the client computing device 102. As shown in FIG. 11, after providing the pet information and the photograph, the pet medicine application 108 determines an appropriate medicine for the pet based on the age of the pet and the weight of the pet. In this case, the user may choose one of a yearly subscription for $84.00 or $8.00 for a monthly subscription of Wags Advance.

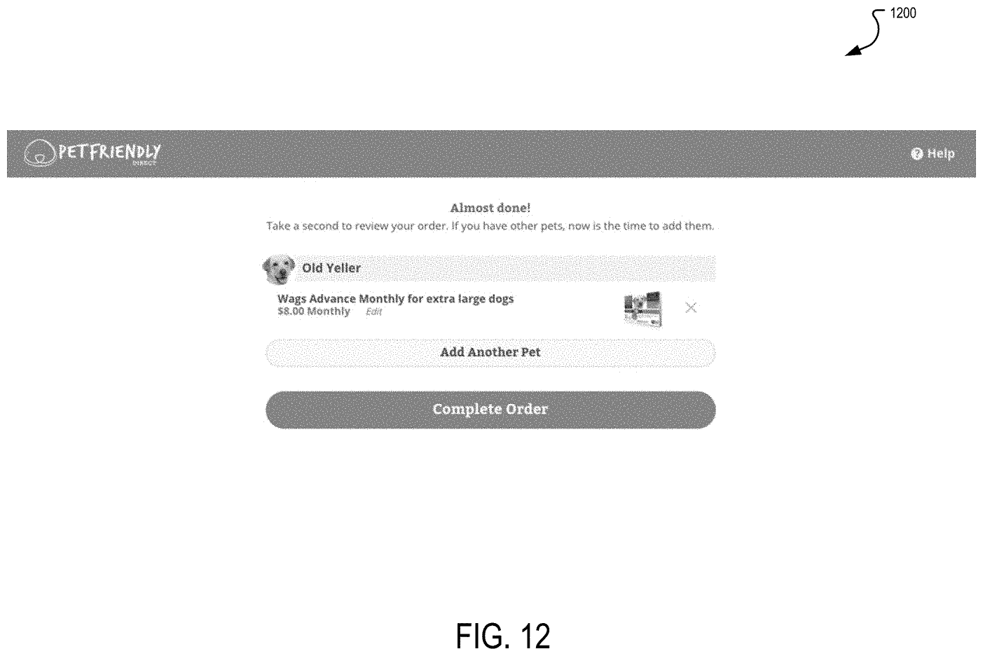

FIG. 12 shows an example user interface 1200 generated by the pet medicine application 108 and transmitted from the server computing device 104 to the client computing device 102 and displayed by the client computing device 102. As shown in FIG. 12, after selecting a monthly subscription for Old Yeller, the user may complete the order.

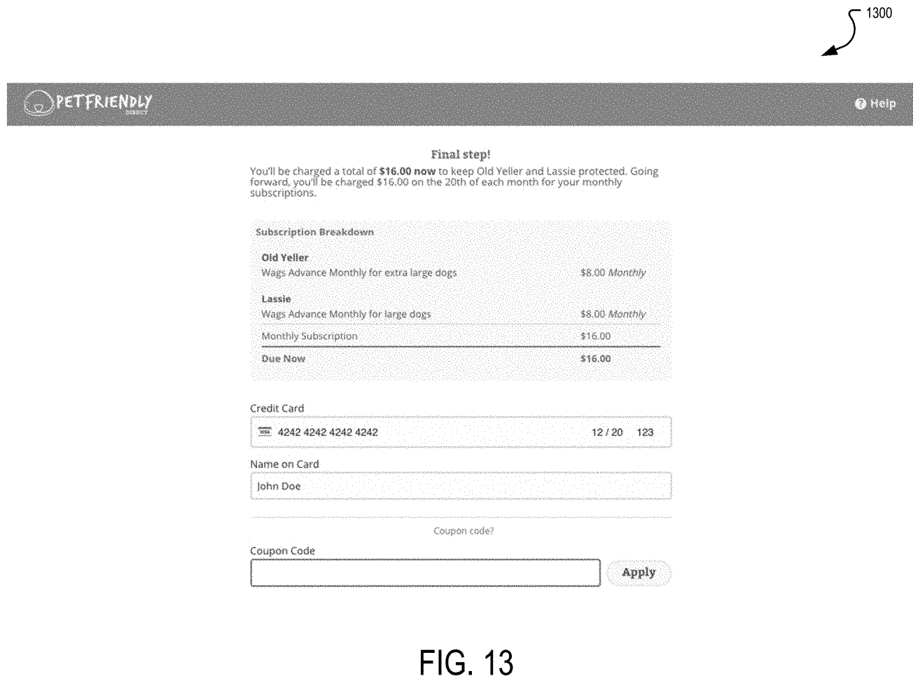

FIG. 13 shows an example user interface 1300 generated by the pet medicine application 108 and transmitted from the server computing device 104 to the client computing device 102 and displayed by the client computing device 102. As shown in FIG. 13, after entering all pet information and selecting a monthly subscription for Old Yeller and a second dog Lassie, the user may provide payment information to complete the order. The user may provide credit card information and a name on the card. In addition, if applicable, the user may provide a coupon code.

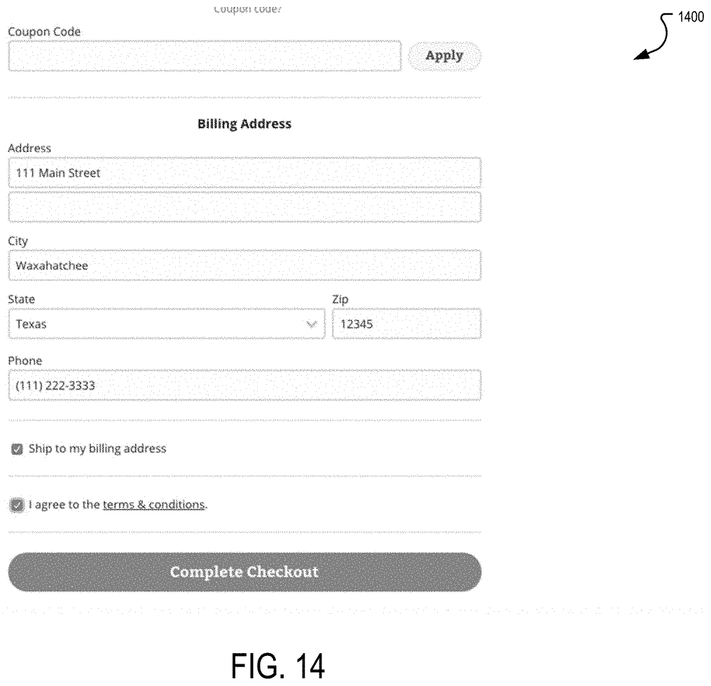

FIG. 14 shows an example user interface 1400 generated by the pet medicine application 108 and transmitted from the server computing device 104 to the client computing device 102 and displayed by the client computing device 102. FIG. 14 shows an additional user interface associated with the order process user interface as shown in FIG. 13. In one example, the user interface in FIG. 14 is shown when the user scrolls down from the user interface shown in FIG. 13.

As shown in FIG. 14, the user may provide billing address information and a shipping address if the billing address is different than the shipping address. In addition, the user may agree to the terms and conditions. When the user selects a complete checkout user interface element, the server computing device 104 may process the order and charge the card associated with the order.

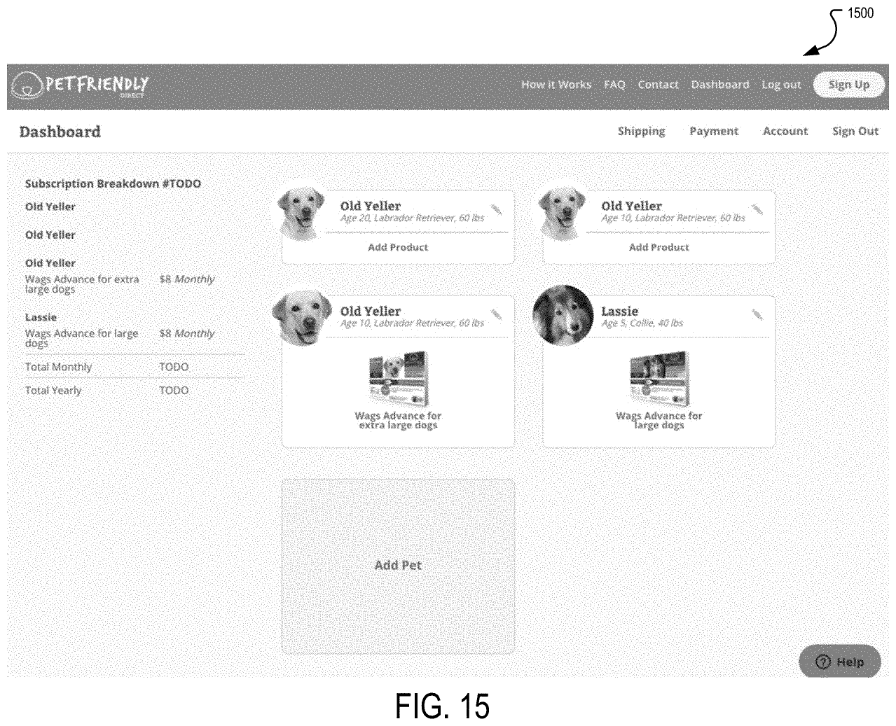

FIG. 15 shows an example user interface 1500 generated by the pet medicine application 108 and transmitted from the server computing device 104 to the client computing device 102 and displayed by the client computing device 102. As shown in FIG. 15, the user may view a dashboard associated with the user account. FIG. 15 shows a first user interface element that is associated with Old Yeller and an image of the pet medicine and associated pet medicine box for Old Yeller. In addition, FIG. 15 shows a second user interface element that is associated with Lassie and an image of the pet medicine and associated pet medicine box for Lassie.

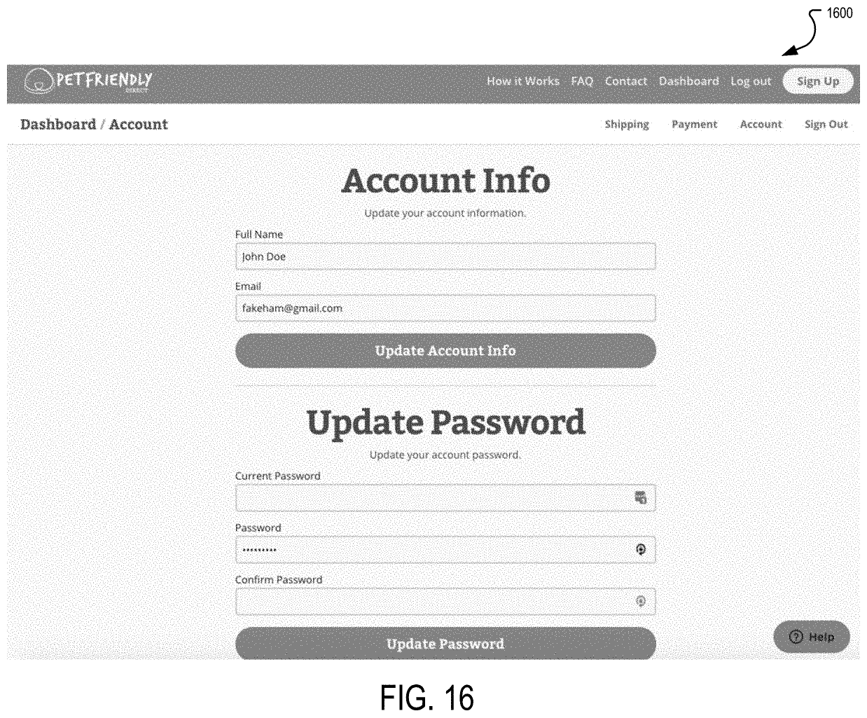

FIG. 16 shows an example user interface 1600 generated by the pet medicine application 108 and transmitted from the server computing device 104 to the client computing device 102 and displayed by the client computing device 102. FIG. 16 shows account information associated with the user account and allows the user to update the account information. In addition, the user may update an account password associated with the user account.

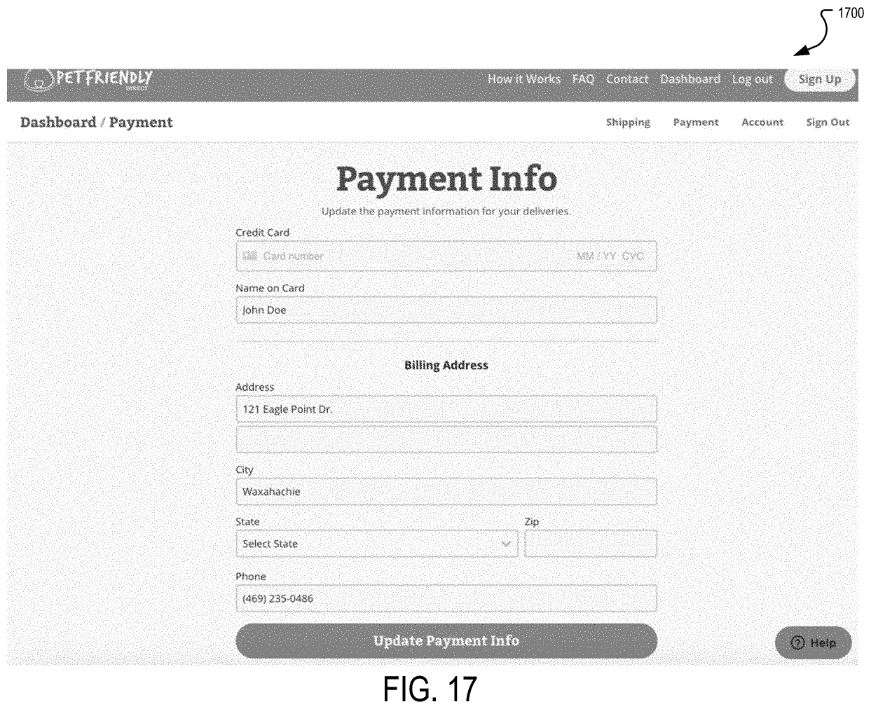

FIG. 17 shows an example user interface 1700 generated by the pet medicine application 108 and transmitted from the server computing device 104 to the client computing device 102 and displayed by the client computing device 102. FIG. 17 shows payment information associated with the user account and deliveries. In addition, FIG. 17 shows a billing address associated with the user account and allows the user to modify and/or correct the billing address.

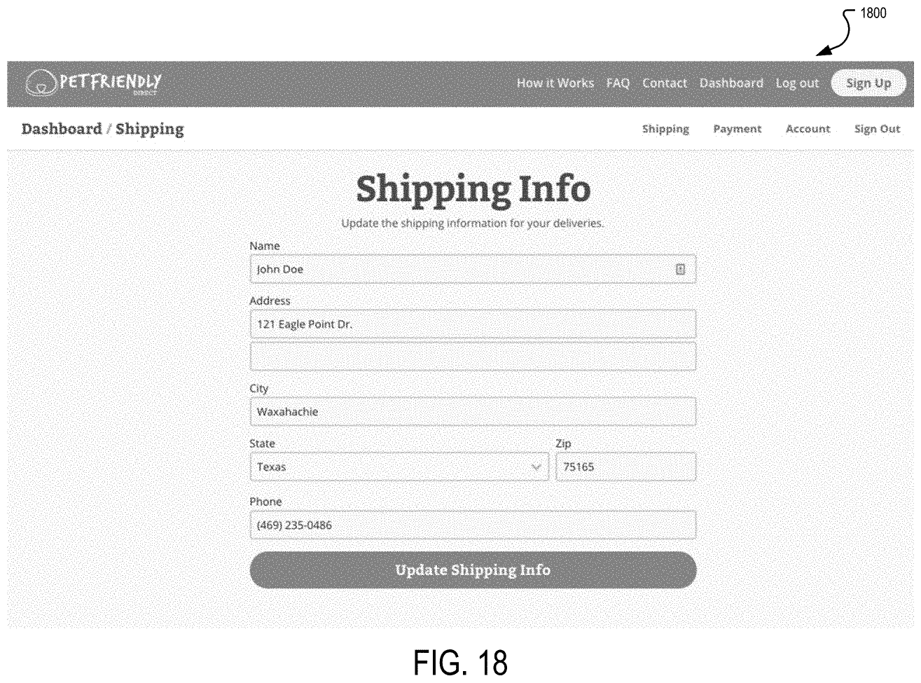

FIG. 18 shows an example user interface 1800 generated by the pet medicine application 108 and transmitted from the server computing device 104 to the client computing device 102 and displayed by the client computing device 102. FIG. 18 shows a shipping address associated with the user account and allows the user to modify and/or correct the shipping address.

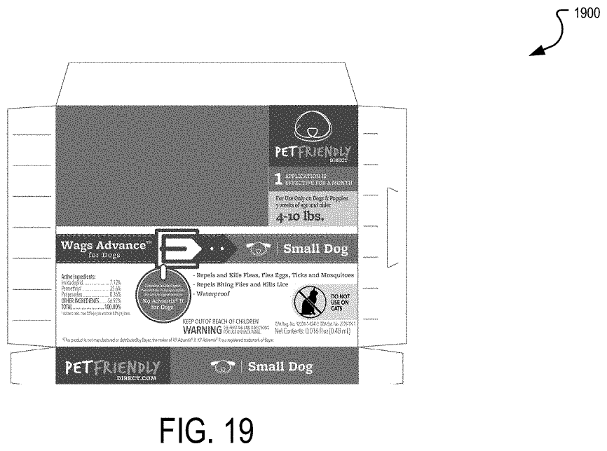

FIG. 19 shows an example pet medicine box 1900 that may be generated by the server computing device 104. As shown in FIG. 19, the pet medicine box 1900 includes an area for the personalized pet label and indicates that it is for a small dog of four to ten pounds. The pet medicine box may be green or another color. The personalized pet label may be placed on, embedded to, and/or affixed to the area for the personalized pet label. In another example, the personalized pet label may be printed directly onto the pet medicine box or automatically applied to the pet medicine box.

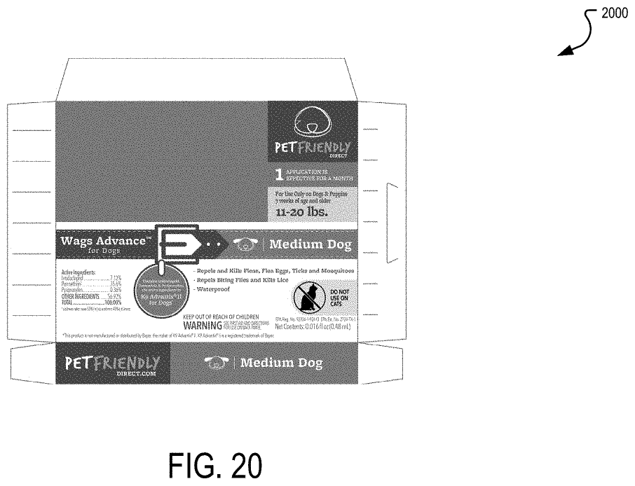

FIG. 20 shows an example pet medicine box 2000 that may be generated by the server computing device 104. As shown in FIG. 20, the pet medicine box 2000 includes an area for the personalized pet label and indicates that it is for a medium dog of eleven to twenty pounds. The pet medicine box may be blue or another color. The personalized pet label may be placed on, embedded to, and/or affixed to the area for the personalized pet label.

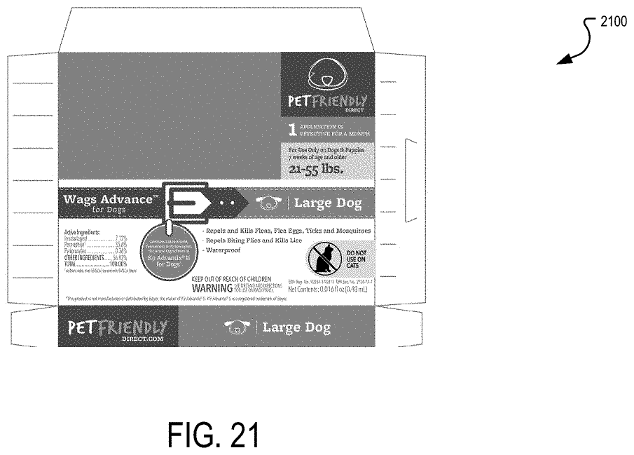

FIG. 21 shows an example pet medicine box 2100 that may be generated by the server computing device 104. As shown in FIG. 21, the pet medicine box 2100 includes an area for the personalized pet label and indicates that it is for a large dog of twenty-one to fifty-five pounds. The pet medicine box may be pink or another color. The personalized pet label may be placed on, embedded to, and/or affixed to the area for the personalized pet label.

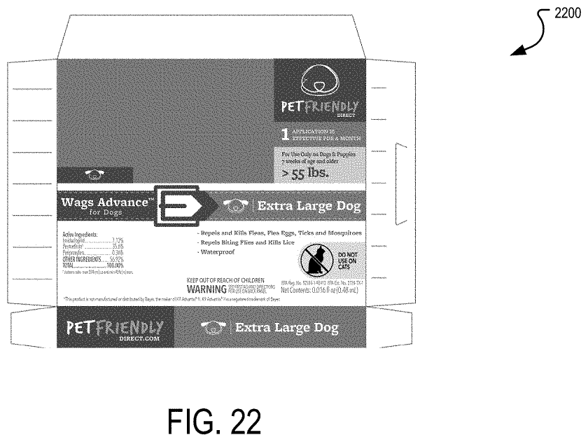

FIG. 22 shows an example pet medicine box 2200 that may be generated by the server computing device 104. As shown in FIG. 22, the pet medicine box 2200 includes an area for the personalized pet label and indicates that it is for an extra large dog of greater than fifty-five pounds. The pet medicine box may be purple or another color. The personalized pet label may be placed on, embedded to, and/or affixed to the area for the personalized pet label.

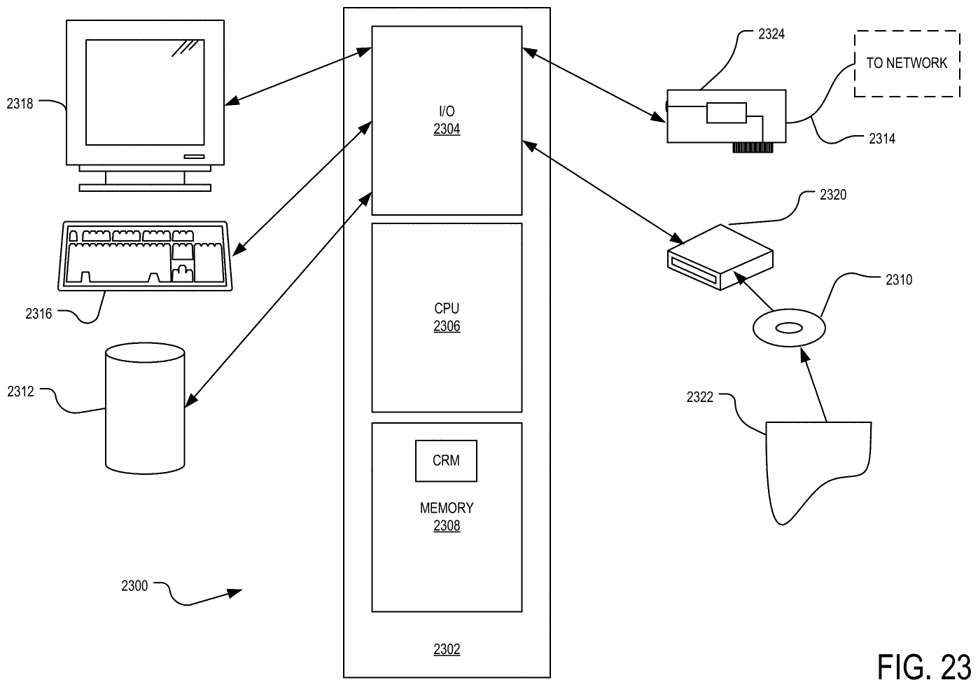

FIG. 23 illustrates an example computing system 2300 that may implement various systems, such as the client computing device 102 and the server computing device 104, and the methods discussed herein, such as process 300 and process 400. A general purpose computer system 2300 is capable of executing a computer program product to execute a computer process. Data and program files may be input to the computer system 2300, which reads the files and executes the programs therein such as the pet medicine application 108. Some of the elements of a general purpose computer system 2300 are shown in FIG. 23 wherein a processor 2302 is shown having an input/output (I/O) section 2304, a central processing unit (CPU) 2306, and a memory section 2308. There may be one or more processors 2302, such that the processor 2302 of the computer system 2300 comprises a single central-processing unit 2306, or a plurality of processing units, commonly referred to as a parallel processing environment. The computer system 2300 may be a conventional computer, a server, a distributed computer, or any other type of computer, such as one or more external computers made available via a cloud computing architecture. The presently described technology is optionally implemented in software devices loaded in memory 2308, stored on a configured DVD/CD-ROM 2310 or storage unit 2312, and/or communicated via a wired or wireless network link 2314, thereby transforming the computer system 2300 in FIG. 23 to a special purpose machine for implementing the described operations.

The memory section 2308 may be volatile media, nonvolatile media, removable media, non-removable media, and/or other media or mediums that can be accessed by a general purpose or special purpose computing device. For example, the memory section 2308 may include non-transitory computer storage media and communication media. Non-transitory computer storage media further may include volatile, nonvolatile, removable, and/or non-removable media implemented in a method or technology for the storage (and retrieval) of information, such as computer/machine-readable/executable instructions, data and data structures, engines, program modules, and/or other data. Communication media may, for example, embody computer/machine-readable/executable, data structures, program modules, algorithms, and/or other data. The communication media may also include an information delivery technology. The communication media may include wired and/or wireless connections and technologies and be used to transmit and/or receive wired and/or wireless communications.

The I/O section 2304 is connected to one or more user-interface devices (e.g., a keyboard 2316 and a display unit 2318), a disc storage unit 2312, and a disc drive unit 2320. Generally, the disc drive unit 2320 is a DVD/CD-ROM drive unit capable of reading the DVD/CD-ROM medium 2310, which typically contains programs and data 2322. Computer program products containing mechanisms to effectuate the systems and methods in accordance with the presently described technology may reside in the memory section 2304, on a disc storage unit 2312, on the DVD/CD-ROM medium 2310 of the computer system 2300, or on external storage devices made available via a cloud computing architecture with such computer program products, including one or more database management products, web server products, application server products, and/or other additional software components. Alternatively, a disc drive unit 2320 may be replaced or supplemented by another storage medium drive unit. The network adapter 2324 is capable of connecting the computer system 2300 to a network via the network link 2314, through which the computer system can receive instructions and data. Examples of such systems include personal computers, Intel or PowerPC-based computing systems, AMD-based computing systems, ARM-based computing systems, and other systems running a Windows-based, a UNIX-based, or other operating system. It should be understood that computing systems may also embody devices such as Personal Digital Assistants (PDAs), mobile phones, tablets or slates, multimedia consoles, gaming consoles, set top boxes, etc.

When used in a LAN-networking environment, the computer system 2300 is connected (by wired connection and/or wirelessly) to a local network through the network interface or adapter 2324, which is one type of communications device. When used in a WAN-networking environment, the computer system 2300 typically includes a modem, a network adapter, or any other type of communications device for establishing communications over the wide area network. In a networked environment, program modules depicted relative to the computer system 2300 or portions thereof, may be stored in a remote memory storage device. It is appreciated that the network connections shown are examples of communications devices for and other means of establishing a communications link between the computers may be used.

In an example implementation, source code executed by the client computing device 102, the server computing device 104, a plurality of internal and external databases, source databases, and/or cached data on servers are stored in memory 114 of the client computing device 102, memory 124 of the server computing device 104, or other storage systems, such as the disk storage unit 2312 or the DVD/CD-ROM medium 2310, and/or other external storage devices made available and accessible via a network architecture. The source code executed by the client computing device 102 and the server computing device 104 may be embodied by instructions stored on such storage systems and executed by the processor 2302.

Some or all of the operations described herein may be performed by the processor 2302, which is hardware. Further, local computing systems, remote data sources and/or services, and other associated logic represent firmware, hardware, and/or software configured to control operations of the automatic pet medicine system 100 and/or other components. Such services may be implemented using a general purpose computer and specialized software (such as a server executing service software), a special purpose computing system and specialized software (such as a mobile device or network appliance executing service software), or other computing configurations. In addition, one or more functionalities disclosed herein may be generated by the processor 2302 and a user may interact with a Graphical User Interface (GUI) using one or more user-interface devices (e.g., the keyboard 2316, the display unit 2318, and the user devices 2304) with some of the data in use directly coming from online sources and data stores. The system set forth in FIG. 23 is but one possible example of a computer system that may employ or be configured in accordance with aspects of the present disclosure.

In the present disclosure, the methods disclosed may be implemented as sets of instructions or software readable by a device. Further, it is understood that the specific order or hierarchy of steps in the methods disclosed are instances of example approaches. Based upon design preferences, it is understood that the specific order or hierarchy of steps in the method can be rearranged while remaining within the disclosed subject matter. The accompanying method claims present elements of the various steps in a sample order, and are not necessarily meant to be limited to the specific order or hierarchy presented.

The described disclosure may be provided as a computer program product, or software, that may include a non-transitory machine-readable medium having stored thereon executable instructions, which may be used to program a computer system (or other electronic devices) to perform a process according to the present disclosure. A non-transitory machine-readable medium includes any mechanism for storing information in a form (e.g., software, processing application) readable by a machine (e.g., a computer). The non-transitory machine-readable medium may include, but is not limited to, magnetic storage medium, optical storage medium (e.g., CD-ROM); magneto-optical storage medium, read only memory (ROM); random access memory (RAM); erasable programmable memory (e.g., EPROM and EEPROM); flash memory; or other types of medium suitable for storing electronic executable instructions.

The description above includes example systems, methods, techniques, instruction sequences, and/or computer program products that embody techniques of the present disclosure. However, it is understood that the described disclosure may be practiced without these specific details.

It is believed that the present disclosure and many of its attendant advantages will be understood by the foregoing description, and it will be apparent that various changes may be made in the form, construction and arrangement of the components without departing from the disclosed subject matter or without sacrificing all of its material advantages. The form described is merely explanatory, and it is the intention of the following claims to encompass and include such changes.

While the present disclosure has been described with reference to various embodiments, it will be understood that these embodiments are illustrative and that the scope of the disclosure is not limited to them. Many variations, modifications, additions, and improvements are possible. More generally, embodiments in accordance with the present disclosure have been described in the context of particular implementations. Functionality may be separated or combined in blocks differently in various embodiments of the disclosure or described with different terminology. These and other variations, modifications, additions, and improvements may fall within the scope of the disclosure as defined in the claims that follow.

What is claimed is:

1. A system comprising:
a memory; and
at least one processor to:
receive a photograph of a pet and store the photograph of the pet in a database;
automatically determine at least one pet medicine subscription that is ready to ship based on an estimated delivery date by iterating over a list of pet medicine subscriptions;
begin processing an automatic shipment associated with the at least one pet medicine subscription and retrieve shipment data associated with the at least one pet medicine subscription from the database;
transmit the shipment data associated with the at least one pet medicine subscription to a server computing device;
receive a shipment label from the server computing device, the shipment label associated with the shipment;
generate a personalized pet label for the at least one pet medicine subscription, the personalized pet label having the photograph of the pet; and
transmit a label sheet having the personalized pet label and the shipping label.

2. The system of claim 1, the at least one processor further to receive user input from a client computing device, the user input comprising pet medicine subscription information for the pet having a particular pet medicine subscription comprising a pet name, a breed selected from a list of breeds, a pet weight, a pet age, a pet home state, an email address, and a subscription option comprising one of yearly and monthly.

3. The system of claim 2, the at least one processor further to generate the personalized pet label, the personalized pet label comprising the photograph of the pet, the pet name, the pet weight, and the pet age.

4. The system of claim 1, the at least one processor further to transmit the label sheet to a customer service client computing device.

5. The system of claim 1, the at least one processor further to transmit the label sheet to a printer to print the label sheet.

6. The system of claim 1, the at least one processor further to transmit the shipment data to the server computing device using a web application programming interface (API).

7. The system of claim 1, the at least one processor further to transmit a dashboard user interface to a client computing device, the dashboard user interface having at least one user interface element that comprises the personalized pet label on a pet medicine box.

8. A method comprising:
receiving, by at least one processor, a photograph of a pet and storing the photograph of the pet in a database,
automatically determining, by the at least one processor, at least one pet medicine subscription that is ready to ship based on an estimated delivery date;
begin processing, by the at least one processor, an automatic shipment associated with the at least one pet medicine subscription and retrieving shipment data associated with the at least one pet medicine subscription from the database;
transmitting, by the at least one processor, the shipment data associated with the at least one pet medicine subscription to a server computing device;
receiving, by the at least one processor, a shipment label from the server computing device, the shipment label associated with the shipment;
generating, by the at least one processor, a personalized pet label for the at least one pet medicine subscription, the personalized pet label having the uploaded photograph of the pet; and
transmitting, by the at least one processor, a label sheet having the personalized pet label and the shipping label.

9. The method of claim 8, further comprising receiving user input from a client computing device, the user input comprising pet medicine subscription information for the pet having a particular pet medicine subscription comprising a pet name, a breed selected from a list of dog breeds, a pet weight, a pet age, a pet home state, an email address, and a subscription option comprising one of yearly and monthly.

10. The method of claim 9, further comprising generating the personalized pet label, the personalized pet label comprising the photograph of the pet, the pet name, the pet weight, and the pet age.

11. The method of claim 8, further comprising transmitting the label sheet to a customer service client computing device.

12. The method of claim 8, further comprising transmitting the label sheet to a printer to print the label sheet.

13. The method of claim 8, further comprising transmitting the shipment data to the server computing device using a web application programming interface (API).

14. The method of claim 8, further comprising transmitting a dashboard user interface to a client computing device, the dashboard user interface having at least one user interface element that comprises the personalized pet label on a pet medicine box.

15. A non-transitory computer-readable storage medium, having instructions stored thereon that, when executed by a computing device cause the computing device to perform operations, the operations comprising:
receiving a photograph of a pet and storing the photograph of the pet in a database;
automatically determining at least one pet medicine subscription that is ready to ship based on an estimated delivery date;
begin processing an automatic shipment associated with the at least one pet medicine subscription and retrieving shipment data associated with the at least one pet medicine subscription from the database;
transmitting the shipment data associated with the at least one pet medicine subscription to a server computing device;
receiving a shipment label from the server computing device, the shipment label associated with the shipment;
generating a personalized pet label for the at least one pet medicine subscription, the personalized pet label having the photograph of the pet; and
transmitting a label sheet having the personalized pet label and the shipping label.

16. The non-transitory computer-readable storage medium of claim 15, the operations further comprising receiving user input from a client computing device, the user input comprising pet medicine subscription information for the pet having a particular pet medicine subscription comprising a pet name, a breed selected from a list of breeds, a pet weight, a pet age, a pet home state, an email address, and a subscription option comprising one of yearly and monthly.

17. The non-transitory computer-readable storage medium of claim 16, the operations further comprising generating the personalized pet label, the personalized pet label comprising the photograph of the pet, the pet name, the pet weight, and the pet age.

18. The non-transitory computer-readable storage medium of claim 15, the operations further comprising transmitting the label sheet to a customer service client computing device.

19. The non-transitory computer-readable storage medium of claim 15, the operations further comprising transmitting the label sheet to a printer to print the label sheet.

20. The non-transitory computer-readable storage medium of claim 15, the operations further comprising transmitting the shipment data to the server computing device using a web application programming interface (API).

21. A system comprising:
a memory comprising executable instructions; and
at least one processor to execute the instructions to:
electronically receive, from an owner of a particular pet, a photograph of the particular pet and pet identifying information for the particular pet and store the photograph of the particular pet and the pet identifying information for the particular pet in a database along with information identifying the owner;
automatically determine at least one pet medicine for the particular pet is to be shipped;
retrieve shipment data associated with the particular pet from the database;
generate a personalized pet label for the at least one pet medicine, the personalized pet label having the photograph of the particular pet and the pet identifying information for the particular pet;
apply the personalized pet label to the at least one pet medicine for shipping to the owner based on the shipment data; and
ship the at least one pet medicine with the personalized pet label to the owner based on the shipment data.

22. The system of claim 21, wherein the pet identifying information for the particular pet comprises one or more of a pet name, a pet breed, a pet weight, a pet age, and an address of the pet.

23. The system of claim 21, the at least one processor further to receive user input from a client computing device, the user input comprising information for the at least one pet medicine.

24. The system of claim 21, the at least one processor further to receive the pet identifying information from a client computing device, the pet identifying information comprising one or more of a pet name, a pet breed, a pet weight, a pet age, and an address of the pet.

25. The system of claim 21, the at least one processor further to:
electronically receive, from the owner of a plurality of pets, a photograph corresponding to each pet and pet identifying information corresponding to each pet and store the photograph and the pet identifying information corresponding to each pet in the database along with information identifying the owner;
automatically determine at least one pet medicine for each pet is to be shipped;
retrieve shipment data associated with each pet from the database;
generate a personalized pet label for each pet medicine, each personalized pet label having the photograph and the pet identifying information for a corresponding pet;
apply each personalized pet label to the pet medicine for the corresponding pet for shipping to the owner based on the shipment data; and
ship each pet medicine with the corresponding personalized pet label to the owner based on the shipment data.

26. A method comprising:
electronically receiving, by at least one processor, from an owner of a particular pet, a photograph of the particular pet and pet identifying information for the particular pet and storing the photograph of the particular pet and the pet identifying information for the particular pet in a database along with information identifying the owner;
automatically determining, by the at least one processor, at least one pet medicine for the particular pet is to be shipped;
retrieving, by the at least one processor, shipment data associated with the particular pet from the database;
generating, by the at least one processor, a personalized pet label for the at least one pet medicine, the personalized pet label having the photograph of the particular pet and the pet identifying information for the particular pet;
applying, by the at least one processor, the personalized pet label to the at least one pet medicine for shipping to the owner based on the shipment data; and
shipping, by the at least one processor, the at least one pet medicine with the personalized pet label to the owner based on the shipment data.

27. The method of claim 26, wherein the pet identifying information for the particular pet comprises one or more of a pet name, a pet breed, a pet weight, a pet age, and an address of the pet.

28. The method of claim 26, further comprising receiving user input from a client computing device, the user input comprising information for the at least one pet medicine.

29. The method of claim 26, further comprising receiving the pet identifying information from a client computing device, the pet identifying information comprising one or more of a pet name, a pet breed, a pet weight, a pet age, and an address of the pet.

30. The method of claim 26, further comprising:
electronically receiving, from the owner of a plurality of pets, a photograph corresponding to each pet and pet identifying information corresponding to each pet and storing the photograph and the pet identifying information corresponding to each pet in the database along with information identifying the owner;
automatically determining at least one pet medicine for each pet is to be shipped;
retrieving shipment data associated with each pet from the database;
generating a personalized pet label for each pet medicine, each personalized pet label having the photograph and the pet identifying information for a corresponding pet;
applying each personalized pet label to the pet medicine for the corresponding pet for shipping to the owner based on the shipment data; and
shipping each pet medicine with the corresponding personalized pet label to the owner based on the shipment data.
